(19) United States
(12) Patent Application Publication
(10) Pub. No.: US 2010/0324290 A1
(43) Pub. Date: Dec. 23, 2010

(54) CRYSTALLINE FORM I OF LAMIVUDINE AND ITS PREPARATION

(75) Inventors: Sayeed Mukhtar, West Singhbum (IN); Vishwesh Pravinchandra Pandya, Jamnagar (IN); Shivanand Shrishailacpa Kokatnur, Bijapur (IN)

Correspondence Address: Ranbaxy Inc., Intellectual Property Department, 600 College Road East, PRINCETON, NJ, 08540, US

(73) Assignee: RANBAXY LABORATORIES LIMITED, New Delhi, Delhi (IN)

(21) Appl. No.: 12/745,033

(22) PCT Filed: Apr. 30, 2008
(86) PCT No.: PCT/IB2008/051692

§ 371 Date: Jul. 16, 2010

(30) Foreign Application Priority Data
Nov. 29, 2007 (IN) 2501/DEL/2007

(51) Int. Cl. C07D 417/04

(52) U.S. Cl. 544/317

(57) ABSTRACT

The present invention relates to a stable crystalline Form I of lamivudine. The present invention further relates to a process for the preparation of the stable crystalline Form (I) of the stable crystalline Form (I) of lamivudine.

### FIELD OF THE INVENTION

The present invention relates to a stable crystalline Form I of lamivudine. The present invention further relates to a process for the preparation of the stable crystalline Form I of lamivudine.

### BACKGROUND OF THE INVENTION

Lamivudine is a substituted 1,3-oxathiolane and it is presently available in the market as an antiretroviral agent. Lamivudine is a cis-(−)-isomer and it is chemically (2R,cis)-4-amino-1-(2-hydroxymethyl-1,3-oxathiolan-5-yl)-(1H)-pyrimidin-2-one of Formula I (A) having the structure as depicted below.

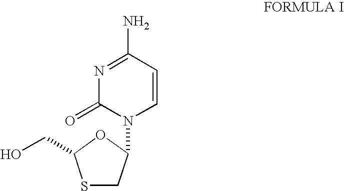

U.S. Pat. No. 5,905,082 provides a process for preparing lamivudine by enzymatic separation of (±)-cis-4-amino-1-(2-hydroxymethyl-1,3-oxathiolan-5-yl)-(1H)-pyrimidine-2-one. However, according to U.S. '082 patent, lamivudine so obtained has an enantiomeric excess of only about 90% and it is referred as “Intermediate 5”. The Intermediate 5 is dissolved in water by heating to 45° C. and cooled to 30° C. The solid product crystallized as unstirrable mass is broken up, stirred at 10° C., filtered and washed two times with industrial methylated spirit. The washed material is dried and the product obtained is referred as Form I. However, U.S. '082 patent is silent about rate of cooling of aqueous solution containing Intermediate 5. The Form I so obtained is further suspended in industrial methylated spirit, stirred at 50° C. for 1 h and a small amount of sample is removed. The remaining mixture is dried under vacuum at 50° C. and the product obtained is referred as 100% Form II. U.S. '082 patent also provides a process of converting Form I to Form II in industrial methylated spirit by seeding.

J. Pharm. Sci., (1996), 85 (2): 193-199 says that Form I is prepared by dissolving lamivudine in hot water, adding an equal volume of methanol and cooling in a refrigerator. However, J. Pharm. Sci., (1996), 85 (2):193-199 does not disclose any method to obtain starting substrate of lamivudine, and also the crystallization conditions and quantities. J. Chem. Soc., Perkin Trans., (1997), 2: 2653-2659 says that Form I is crystallized as needles from solutions in water, methanol or aqueous alcohols whereas Form II is obtained as tetragonal bipyramids on slow recrystallization from dry ethanol, n-propanol or mixtures of ethanol and less polar organic solvents. However, J. Chem. Soc., Perkin Trans. (1997), 2: 2653-2659 also does not disclose any method to obtain starting substrate of lamivudine, and also the crystallization conditions and quantities. However, these references provide characterization methods of Form I and Form II by XRPD, DSC, Scanning Electron Micrographs and single crystal analysis.

U.S. Pat. No. 6,329,522 provides a process for purification of lamivudine by the formation of salicylate salt and a crystallization method for lamivudine from isopropyl acetate. However, both the preparation of lamivudine salicylate and crystallization of lamivudine involve seeding, and U.S. '522 patent does not disclose any method to obtain the seed crystals of lamivudine salicylate as well as lamivudine. WO 03/027106 provides a process for preparing Form II of lamivudine from lamivudine salicylate using ethyl acetate and acetonitrile as solvents and triethylamine as a base. However, WO '106 application does not disclose any specific method to obtain lamivudine salicylate, which is the starting material.

WO 2007/119248 provides a process for the preparation of Form III of lamivudine. Form III is prepared by dissolving Form II in water by heating to 45° C. and subsequent cooling to 30° C. However, the time involved for reducing the temperature from 45° C. to 30° C. varies form 15 minutes to 1 hour 40 minutes. The mixture so obtained is further stirred at 10° C. for 1 hour, filtered and dried in vacuum at 45° C. for 24 hours to obtain Form III. The process also involves optional washing with ethanol or industrial methylated spirit. Form III is also prepared by dissolving Form I in water by heating to 45° C. and subsequent cooling to 10° C. However, the time involved for reducing the temperature from 45° C. to 10° C. is 10 minutes. The mixture so obtained is stirred at 10° C. for 1 hour, filtered and dried in vacuum at 45° C. for 24 hours to obtain Form III. Form III is also prepared by stirring a suspension of Form I or Form II in water at 25° C. for 24 h or 48 hours. The mixture is further stirred at 10° C. for 1 hour, filtered and dried in vacuum at 45° C. for 24 hours to obtain Form III.

WO 2007/119248 application also provides processes for preparing Form I and Form II of lamivudine. Form I is prepared by dissolving lamivudine in water by heating to 45° C. and subsequent cooling to 30° C. in 0.5 minute. The solid product crystallized as unstirrable mass is broken up, stirred at 10° C., filtered and washed with industrial methylated spirit. The washed material is dried in vacuum at 45° C. for 24 hours to obtain Form I. Form I is also prepared in a similar way from a mixture of water and denaturated spirit. However, in this process, the time involved for reducing the temperature from 45° C. to 30° C. is 12 minutes and it also involves seeding with Form I crystals. The washing at the final step is also carried out with a mixture of water and denaturated spirit. Form II is prepared refluxing lamivudine in ethanol and partially removing the solvent by distillation. The concentrated solution is cooled to 15° C. in 35 minutes, stirred at 15° C. for 1 hour, filtered and washed with ethanol. The washed product is dried in vacuum at 50° C. for 12 hours to obtain Form II. However, WO '248 application does not disclose any method to obtain starting lamivudine for preparing Form I or Form II.

Both WO '106 application and U.S. '082 patent say that Form I crystals are not favored for pharmaceutical formulation and exhibit poor flow characteristics. WO '106 application and U.S. '082 patent further say that Form I crystals are a less stable polymorphic forms. WO '248 application says that Form I converts to Form II during milling and formulation operation. WO '248 application also mentions that, due to above mentioned issues, Form II is used for formulations.

Though several processes for the preparation of lamivudine Form I have been reported, it has also been reported that Form I is unsuitable for formulation due to handling and stability reasons. Because of this reason, lamivudine Form I had to be converted to Form II before being formulated.

### SUMMARY OF THE INVENTION

We have developed a process for preparing crystalline Form I of lamivudine using water as the only solvent for crystallizing lamivudine. Form I of lamivudine prepared by using such a process is stable does not convert to Form II when subjected to pharmaceutical operations such as milling. Moreover, crystalline Form I of the so prepared remains stable even when it is stored up to three months at a temperature up to about 45° C. and at a relative humidity of about 25% to about 85%. The present process also provides a way to prepare crystalline Form I substantially free of Form II. The stable crystalline Form I of the present invention is suitable for the preparation of pharmaceutical compositions comprising lamivudine.

### BRIEF DESCRIPTION OF THE FIGURES

FIG. 1 is an X-ray powder diffractogram (XRPD) pattern of stable crystalline Form I of lamivudine.

FIG. 2 is an X-ray powder diffractogram (XRPD) pattern of crystalline Form II of lamivudine.

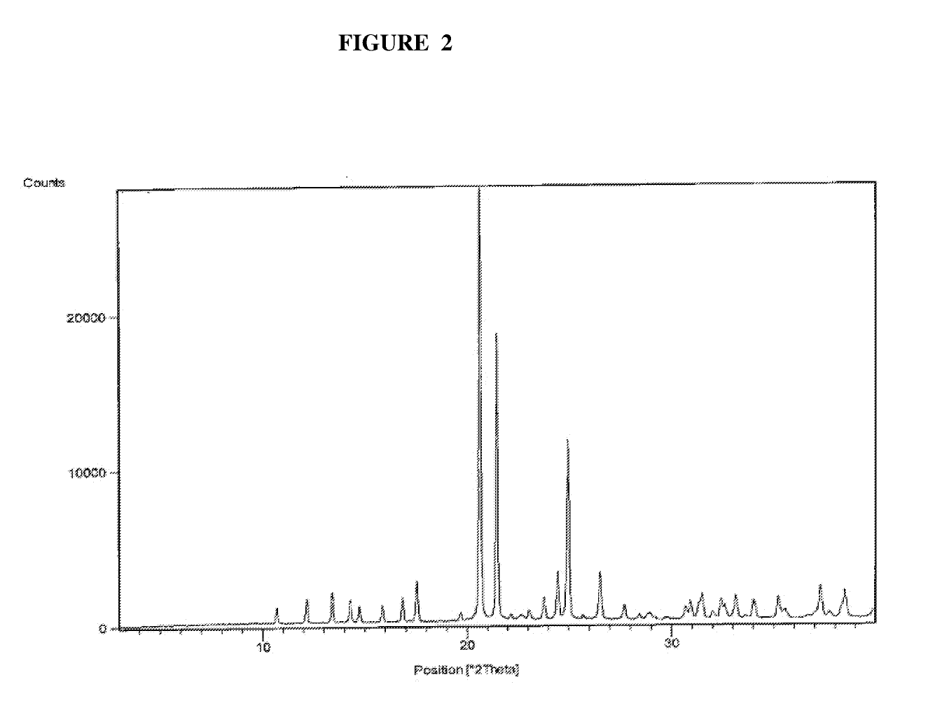

### DETAILED DESCRIPTION OF THE INVENTION

In one aspect, a stable crystalline Form I of lamivudine is provided. The stable crystalline Form I does not convert to Form II or any other solid form by milling. The term milling refers to an act or process of grinding, crushing, pulverizing, powdering, atomizing, pestling, levigating, sifting, sieving, size reducing, or passing through a milling device. The stable crystalline Form I does not convert to Form II or any other solid form when stored at a temperature range of up to about 45° C. and at a relative humidity of about 25% to about 85% up to three months.

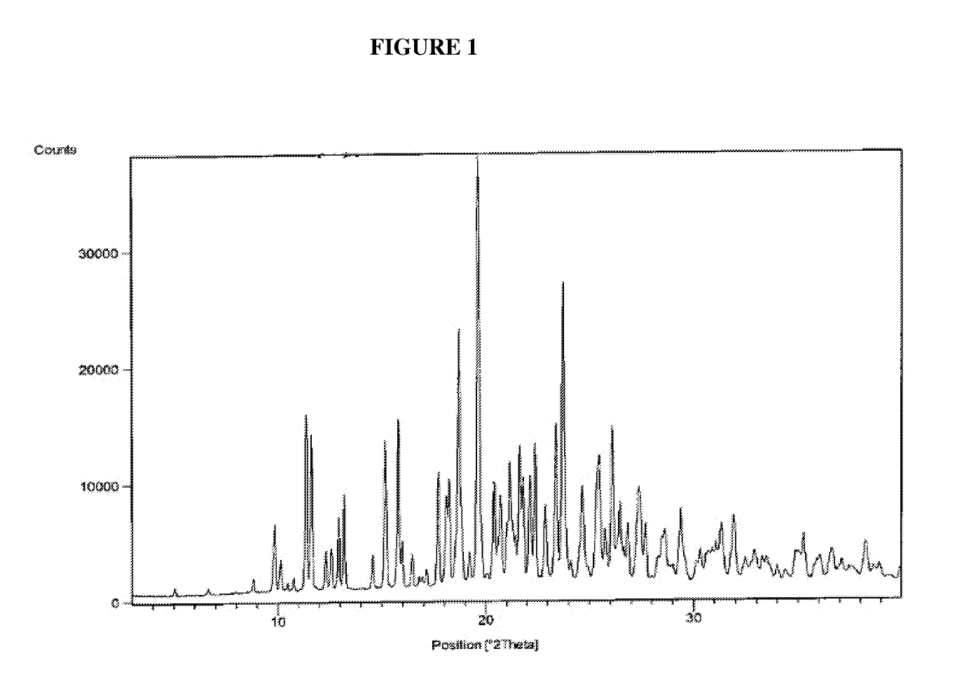

The stable crystalline Form I of lamivudine is characterized by an XRPD pattern substantially as provided in FIG. 1. The XRPD pattern of stable crystalline Form I of lamivudine can be characterized by peaks at 2θ values 9.86, 11.38, 11.63, 13.22, 15.18, 15.81, 17.72, 18.10, 18.24, 18.71, 19.65, 20.40, 20.71, 21.17, 21.64, 21.80, 22.13, 22.38, 22.88, 23.39, 23.71, 24.64, 25.35, 25.45, 26.07, 26.45, 27.35, 27.44 and 29.35±0.2. It is further characterized by additional peaks at 2θ values at 8.83, 10.13, 10.46, 10.76, 12.32, 12.58, 12.94, 14.59, 15.98, 16.47, 16.80, 16.97, 17.16, 18.86, 19.22, 20.06, 21.02, 21.41, 24.10, 24.48, 25.75, 26.83, 27.24, 27.67, 28.28, 28.46, 28.60, 28.98, 30.10, 30.31, 30.57, 30.68, 30.89, 31.05, 31.30, 31.89 and 35.27±0.2. It should be noted that the 2θ values given above are computer-generated and have not been rounded off. It is known that they could be rounded to the nearest decimal.

In another aspect, crystalline Form I of lamivudine substantially free of crystalline Form II of lamivudine is provided. The crystalline Form II of lamivudine is present in the crystalline Form I of present invention in a quantity of about 2% or less, preferably about 1% or less, more preferably about 0.5% or less.

In yet another aspect, a process for the preparation of stable crystalline Form I of lamivudine is provided, wherein the process comprises,
a) dissolving lamivudine in water at a temperature of about 38° C. to about 45° C. to obtain a solution,
b) cooling the solution obtained in step a) to a temperature of about 30° C. or below in about 10 minutes or less to obtain a mixture,
c) stiffing the mixture obtained in step b) at a temperature of about 30° C. or below, and isolating the solid from the mixture thereof, and
d) washing the solid obtained in step c) with water to obtain stable crystalline Form I of lamivudine.

The lamivudine used as the starting material has a chemical purity of about 98% or above and a chiral purity of about 99% or above. The pure lamivudine used as the starting material can exist in any solid form. Preferably pure crystalline Form II of lamivudine is used as the starting material. The pure crystalline Form II of lamivudine can be prepared by reducing (1R,2S,5R)-2-isopropyl-5-methylcyclohexyl (2R,5S)-5-(4-amino-2-oxopyrimidin-1(2H)-yl)-1,3-oxathiolane-2-carboxylate of Formula II,

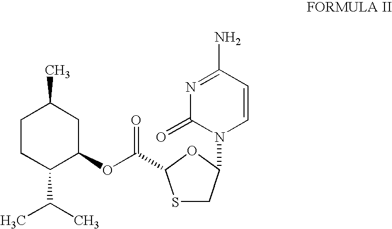

The compound of Formula II can be prepared according to the method provided in U.S. Pat. No. 5,905,082. The compound of Formula II is reduced in the presence of water or an organic solvent or a mixture thereof, to obtain lamivudine. The organic solvent is preferably selected from the group consisting of alkanols, ethers and esters. The organic solvent is more preferably selected from the group consisting of methanol, ethanol, tetrahydrofuran, dioxane, isopropyl acetate and ethyl acetate. The reduction is carried out by using a reducing agent. The reducing agent is preferably sodium borohydride, lithium aluminium hydride, lithium borohydride, lithium-tri-ethyl borohydride or lithium-tri-sec-butyl borohydride. The reducing agent is more preferably sodium borohydride. The reduction is preferably carried out in the presence of a phosphate or borate buffer. The buffer is preferably dipotassium hydrogen phosphate. The lamivudine is further treated with salicylic acid. The lamivudine salicylate is isolated from the reaction mixture without the addition of seed lamivudine salicylate. The isolation of lamivudine salicylate is carried out by stirring the reaction mixture in a temperature range from about 10° C. to about 25° C. The stiffing is preferably carried out initially at about 25° C. to about 30° C. and subsequently at about 10° C. to about 15° C. The stirring can be carried out from about 10 minutes to about 100 hours. The lamivudine salicylate so obtained is treated with a base in the presence of an organic solvent, or a mixture of water and an organic solvent. Preferably a mixture of water and an organic solvent is used as a solvent medium while treating lamivudine salicylate with a base. The organic solvent is preferably selected from the group consisting of alkanols, ethers and esters. The organic solvent is more preferably selected from the group consisting of methanol, ethanol, tetrahydrofuran, dioxane, isopropyl acetate and ethyl acetate. The base is preferably an amine, more preferably a tertiary amine. The treatment of lamivudine salicylate with the base is carried out at a temperature of 55° C. or below, preferably at about 40° C. to about 50° C. The process is accompanied by stirring to facilitate the liberation of lamivudine as a free base. The lamivudine is isolated from the reaction mixture without adding any seed. The isolation is carried out by stiffing the reaction mixture at a temperature of about 0° C. to about 35° C., preferably at about 15° C. to about 30° C., followed by filtration, distillation and/or concentration. A washing of lamivudine with an organic solvent can optionally be employed after isolation.

The lamivudine so obtained can be further purified by dissolving lamivudine in a C₁₋₃ alkanol at reflux temperature and treating the solution with activated charcoal. After removal of the charcoal, lamivudine is obtained as a solid by stiffing the solution at about 0° C. to about 15° C., and the solid is isolated by filtration. The lamivudine can be isolated as a crystalline or amorphous material, including crystalline Form II. The lamivudine so obtained has a chemical purity of about 98% or above and a chiral purity of about 99% or above.

Pure lamivudine is dissolved in water at a temperature of about 38° C. to about 45° C. to obtain a solution. The water employed for dissolving is substantially free of any organic solvent. The solution is cooled to a temperature of about 30° C. or below in about 10 minutes or less to obtain a mixture. The solution is preferably cooled to a temperature of about 25° C. to about 30° C. in about 5 to about 10 minutes. The mixture so obtained is stirred at a temperature of about 30° C. or below to obtain a solid. The stirring is preferably carried out at a temperature of about 5° C. to about 10° C. for about 1 hour. The mixture is filtered to isolate the solid and the solid is washed with water and subsequently dried to obtain stable crystalline Form I of lamivudine. The water employed for washing is substantially free of any organic solvent. The washing is carried out preferably by using water pre-cooled to about 5° C. to 10° C. The drying can be carried out under vacuum at a temperature of about 25° C. to about 45° C., preferably at a temperature of about 35° C. to about 40° C. The stable crystalline Form I of lamivudine so obtained is substantially free of crystalline Form II of lamivudine. The crystalline Form II of lamivudine is present in the stable crystalline Form I so obtained in a quantity of about 2% or less, preferably about 1% or less, more preferably about 0.5% or less.

In still another aspect, a pharmaceutical composition comprising stable crystalline Form I of lamivudine is provided which optionally contains one or more an excipients.

In yet a further aspect, a method of treating HIV or HBV infections is provide which comprises administering to a human in need thereof a therapeutically effective amount of stable crystalline Form I of lamivudine.

XRPD of the samples were obtained by using Panalytical X′Pert Pro X-Ray Powder Diffractometer in the range 3-40 degree 2 theta and under tube voltage and current of 45 Kv and 40 mA respectively. Copper radiation of wavelength 1.54 angstrom and Xceletor detector was used.

While the present invention has been described in terms of its specific embodiments, certain modifications and equivalents will be apparent to those skilled in the art and are intended to be included within the scope of the present invention.

### EXAMPLES

### Example 1

### Preparation of Pure Lamivudine

a) Preparation of lamivudine salicylate:
Dipotassium hydrogen orthophosphate (205.5 g) was added to deionised water (423 mL) and stirred at 25° to 30° C. to obtain a solution. The solution was cooled to 17° to 22° C., followed by the addition of denaturated spirit (900 mL) at the same temperature and stirred for 5 minutes. (1R,2S,5R)-2-isopropyl-5-methylcyclohexyl (2R,5S)-5-(4-amino-2-oxopyrimidin-1(2H)-yl)-1,3-oxathiolane-2-carboxylate (150 g) was added to the mixture at 17° to 22° C. and stirred for 30 minutes at 18° to 20° C. Sodium borohydride solution was added slowly to the reaction mixture over a period of 2 to 3 hours at 18° to 20° C. (Preparation of sodium borohydride solution: Sodium hydroxide (0.75 g) was dissolved in deionised water (143 mL). Sodium borohydride (30 g) was added to the solution at 20° to 35° C., stirred at 20° to 35° C. to obtain a solution and cooled to 17° to 22° C.). The reaction mixture was stirred for 6 hours at 18° to 22° C. and the reaction mixture was allowed to settle at 18° to 25° C. The organic layer was separated and denaturated spirit (150 mL) was added to the aqueous layer at 18° to 25° C. The reaction mixture was stirred for 15 minutes at the same temperature and allowed to settle. The organic layer was separated and combined with the previously obtained organic layer. The pH of the combined organic layer was adjusted to 6.0 to 6.5 with dilute hydrochloric acid (20 mL; prepared by mixing 10 mL of concentrated hydrochloric acid with 10 mL of deionised water) at 18° to 25° C., followed by stirring for 10 minutes at the same temperature. The pH of the reaction mixture was adjusted to 8.0 to 8.5 with aqueous sodium hydroxide solution (28 mL; prepared by dissolving 2.1 g of sodium hydroxide in 27 mL of deionised water) at 18° to 25° C. The reaction mixture was concentrated under vacuum at about 55° C. till the residual volume was about 375 mL. Deionised water (300 mL) was added to the concentrated reaction mixture at 25° to 30° C. and stirred for 10 minutes. The reaction mixture was washed with toluene (2×150 mL) at 25° to 30° C. and the toluene layer was extracted with deionised water (150 mL) at 25° to 30° C. The aqueous layers were combined and salicylic acid (57 g) was added at 25° to 30° C. Deionised water (150 mL) was added to the reaction mixture and heated to 78° to 82° C. to get a clear solution. The reaction mixture was cooled to 25° to 30° C. over a period of 2 hours and stirred at the same temperature for 4 hours. The reaction mixture was further cooled to 10° to 15° C. and stirred for 2 hours at 10° to 15° C. The solid was filtered, washed with deionised water (150 mL) and dried by suction. The solid so obtained was washed with methanol (90 mL, pre-cooled to 5° to 10° C.) and dried at 45° to 50° C. in hot air oven to obtain the title compound.

### Yield: 132 g

b) Preparation of pure lamivudine:
Lamivudine salicylate (120 g) was added to a mixture of ethyl acetate (720 mL) and water (6 mL) at 25° to 35° C. The reaction mixture was heated to 45° to 50° C., followed by the addition of triethylamine (104.76 g) over 30 minutes at 45° to 50° C. The reaction mixture was stirred for 4 hours at the same temperature and cooled to 25° to 30° C. The reaction mixture was stirred for further 30 minutes at 25° to 30° C., filtered and dried by suction. The solid obtained was washed with ethyl acetate. Ethyl acetate (600 mL) was added to the washed solid and heated to 50° to 55° C. The mixture was stirred at 50° to 55° C. for 15 minutes, cooled to 25° to 30° C. and stirred for further 30 minutes. The solid was filtered at 25° to 30° C., washed with ethyl acetate (60 mL) and dried under vacuum at 45° to 50° C. to obtain lamivudine. Lamivudine (60 g) so obtained was added to absolute alcohol (1.2 L) at 25° to 35° C. The reaction mixture was heated to 75° to 78° C. and stirred to obtain a solution. Activated carbon (6 g) was added to the solution so obtained at 75° to 78° C., stirred for 30 minutes at the same temperature and filtered through Celite bed at the same temperature. The carbon bed was washed with absolute alcohol (60 mL; preheated to 75° to 76° C.) and the reaction mixture was concentrated under vacuum to obtain a volume of about 300 mL. The concentrated reaction mixture was heated to 74° to 76° C., stirred for 15 minutes and ° 10° C. in 1 h time and stirred for 30 minutes. The solid was filtered, washed with absolute alcohol (30 mL, pre-cooled to 5° to 10° C.) and dried under vacuum at 50° to 55° C. to obtain the title compound.

### Yield: 53 g

### HPLC Purity: 99.0%

### Chiral Purity: 99.8%

### Example 2

### Preparation of Stable Crystalline Form I of Lamivudine

Pure lamivudine (100 g) obtained as prepared in Example 1 was dissolved in demineralised water (300 mL) at 38° C. to 45° C. The resultant clear solution was cooled to 25° to 30° C. in 5 to 10 minutes. The resultant mixture was further cooled to 5° to 10° C. in 15 to 20 minutes and stirred for 1 hour at 0° to 10° C. The solid was filtered, washed with water (50 mL) pre-cooled to 5° to 8° C., and dried at 35° to 40° C. under vacuum to obtain the title compound having an XRPD pattern as depicted in FIG. 1.

### Yield: 80 g

Form II of lamivudine: Not detectable (Limit of detection: 0.5% by XRPD)
The stable crystalline Form I of lamivudine obtained from Example 2 was subjected to the following conditions:

| | | | Resultant Material |
|---|---|---|---|
| SN | Material Tested | Conditions | after storage |
| 1. | Form I obtained | Stored at 40 ± 2° C. | Form I |
| | from Example 2 | at 75 ± 5% RH for 1 | |
| | | month | |
| 2. | Form I obtained | Stored at 40 ± 2° C. | Form I |
| | from Example 2 | at 75 ± 5% RH for 2 | |
| | | months | |
| 3. | Form I obtained | Stored at 40 ± 2° C. | Form I |
| | from Example 2 | at 75 ± 5% RH for 3 | |
| | | months | |
| 4. | Form I obtained | Stored at 25 ± 2° C. | Form I |
| | from Example 2 | at 60 ± 5% RH for 3 | |
| | | months | |
| 5. | Form I obtained | Milled in a | Form I |
| | from Example 2 | multimill (2 mm | |
| | | mesh size) | |

We claim:

1. A stable crystalline Form I of lamivudine.

2. The stable crystalline Form I of lamivudine according to claim 1, wherein said crystalline form does not convert to Form II or any other solid form by milling.

3. The stable crystalline Form I of lamivudine according to claim 1, wherein said crystalline form does not convert to Form II or any other solid form when stored at a temperature range of up to about 45° C. and at a relative humidity of about 25% to about 85%.

4. The stable crystalline Form I of lamivudine according to claim 1, wherein said crystalline form does not convert to Form II or any other solid form when stored up to about three months or more.

5. The stable crystalline Form I of lamivudine according to claim 1 has an XRPD pattern characterized by peaks at 2θ values 9.9, 11.4, 11.6, 13.2, 15.18, 15.8, 17.7, 18.1, 18.2, 18.7, 19.7, 20.4, 20.7, 21.2, 21.6, 21.8, 22.1, 22.4, 22.9, 23.4, 23.7, 24.6, 25.4, 25.5, 26.1, 26.5, 27.4, 27.4 and 29.4±0.2.

6. Crystalline Form I of lamivudine substantially free of crystalline Form II of lamivudine.

7. Crystalline Form I of lamivudine according to claim 6 contains crystalline Form II of lamivudine in a quantity of about 2% or less.

8. Crystalline Form I of lamivudine according to claim 6 contains crystalline Form II of lamivudine in a quantity of about 1% or less.

9. A process for the preparation of stable crystalline Form I of lamivudine, comprising:
a) dissolving lamivudine in water at a temperature of about 38° C. to about 45° C. to obtain a solution,
b) cooling the solution obtained in step a) to a temperature of about 30° C. or below in about 10 minutes or less to obtain a mixture,
c) stirring the mixture obtained in step b) at a temperature of about 30° C. or below, and isolating the solid from the mixture thereof, and
d) washing the solid obtained in step c) with water to obtain stable crystalline Form I of lamivudine.

10. A process according to claim 9, wherein the water employed in steps a) to d) is substantially free of any organic solvent.
